(19) United States
(12) Patent Application Publication
(10) Pub. No.: US 2011/0207653 A1
(43) Pub. Date: Aug. 25, 2011

(54) Microparticle Encapsulated Thiol-Containing Polypeptides Together with a Redox Reagent

(76) Inventors: Adrian Raiche, Helena, AL (US); Kevin Burton, Hoover, AL (US)

(21) Appl. No.: 12/968,624

(22) Filed: Dec. 15, 2010

Related U.S. Application Data
(60) Provisional application No. 61/288,654, filed on Dec. 21, 2009.

(51) Int. Cl. A61K 38/02

(52) U.S. Cl. 514/1.1

(57) ABSTRACT

The present invention relates to polymeric microparticles comprising a therapeutic polypeptide along with a redox reagent. Also disclosed are methods of using the microparticles, including methods of using the microparticles for delivery of the therapeutic polypeptide to a subject in need thereof.

### CROSS-REFERENCE TO RELATED APPLICATIONS

This application claims the benefit of priority of U.S. Provisional Application No. 61/288,654, filed Dec. 21, 2009, the entire contents of which are incorporated herein by reference.

### BACKGROUND

Disulfide bonds are physiologically relevant bonds that can form between two thiol (—SH) containing residues. Tertiary and quaternary protein structures are often stabilized or formed by disulfide bonds. Disulfide bonds can also form in solutions or pharmaceutical compositions comprising therapeutically active peptides that contain a thiol residue, such as a cysteine or homocysteine, and particularly in more concentrated solutions of thiol-containing polypeptides.

Disulfide bonds can occur either intramolecularly, for example between two residues on the same protein, or intermolecularly (i.e., between two separate peptides). In a physiological environment, inter- and intramolecular disulfide bonds can break and reform through disulfide exchange reactions. In drug-delivery compositions, disulfide exchange reactions that occur among therapeutically active peptides can be problematic. Such reactions can lead to the breakage of desirable disulfide bonds, e.g., an intramolecular disulfide bond which is critical to the structure and therefore activity of the peptide, and can lead to the formation of undesirable disulfide bonds, e.g., intermolecular disulfide bonds that cause peptide aggregate formation. Accordingly, a need exists for compositions and methods that mitigate the effect of disulfide exchange reactionsin pharmaceutical compositions.

### SUMMARY

Disclosed herein are polymer microparticles comprising: a biodegradable polymer matrix, a therapeutic polypeptide comprising at least one natural or unnatural non-natural thiol (—SH) containing amino acid; and a redox reagent comprising at least one thiol (—SH) residue.

### DETAILED DESCRIPTION

Unless the context requires otherwise, the word “comprise,” or variations such as “comprises” or “comprising,” will be understood to imply the inclusion of a stated integer or step or group of integers or steps but not the exclusion of any other integer or step or group of integers or steps.

The singular forms “a,” “an” and “the” include plural referents unless the context clearly dictates otherwise. Thus, for example, reference to “a therapeutic polypeptide” includes mixtures of two or more such agents, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

The term “microparticle” refers to particles that have sizes in the range of about 10 nanometers (nm) to about 2 mm (millimeters), including particles sometimes refered to as nanoparticles, microspheres, nanospheres, microcapsules, and nanocapsules. As such, the term microparticle refers to particles having a variety of internal structure and organizations including homogeneous matrices such as microspheres (and nanospheres) or heterogeneous core-shell matrices (such as microcapsules and nanocapsules), porous particles, multi-layer particles, among others. In a further aspect, the microparticle can have a diameter in the range of from 2 μm to 50 μm, from 2 μm to 30 μm, from 2 μm to 20 μm, or from 2 μm to 10 μm.

When the therapeutic polypeptide or redox reagent is said to be “encapsulated” within the biodegradable polymer matrix, the oligopeptide or redox reagent is within a void defined by the polymer matrix of the microparticle, and/or actually in the polymer matrix (e.g., dissolved or dispersed) itself.

As used herein, the term “therapeutic polypeptide” means any peptide-linked chain of at least 2 natural or non-natural amino acids that is therapeutically useful. The term “polypeptide” includes oligopeptides and proteins. The term “polypeptide” also includes oligopeptides and proteins that have been modified, for example post-translationally modified such as through glycoslylation or phosphorylation of the protein.

The microparticle comprises a biodegradable polymer matrix. The polymer of the matrix can be any biodegradable polymer used in the encapsulation art. The polymers can be homopolymers or copolymers, including block or blocky co- or ter- polymers, random co- or ter- polymers, star polymers, or dendrimers. Any desired molecular weight polymer can be used, depending on the desired properties of the microparticle. If a high strength polymer is desired, then high molecular weight polymers can be used, for example, to meet strength requirements. In other aspects, low or medium molecular weight polymers can be used when, for example, when resorption time of the polymer, rather than microparticle strength is desired.

The molecular weight of the polymer can be important given that molecular weight influences the biodegradation rate of a biodegradable polymer. For a diffusional mechanism of release, the polymer should remain intact until all of the therapeutic polypeptide is released from the polymer and then degrade. The therapeutic polypeptide can also be released from the polymer as the polymer bioerodes. By an appropriate selection of polymeric materials, a polymer formulation can be made such that the resulting polymer exhibits both diffusional release and biodegradation release properties. Molecular weights can be measured by methods known in the art, including gel permeation chromatography, viscosity, light-scattering, among other methods.

The polymer can be formulated so as to degrade within a desired time interval, once present in a subject, or a biological medium. In some aspects, the time interval can be from about less than one day to about 1 month. Longer time intervals can extend to 6 months, including for example, polymers that degrade from about ≧0 to about 6 months, or from about 1 to about 6 months. In other aspects, the polymer can degrade in longer time intervals, up to 2 years or longer, including, for example, from about ≧0 to about 2 years, or from about 1 month to about 2 years. A sustained release formulation of the microparticle and therapeutic polypeptide can deliver the therapeutic polypeptide to a subject over any of these time periods and under a wide variety of release profiles.

The desired therapeutic polypeptide release mechanism can influence the selection of the polymer. A biocompatible polymer, for example, can be selected so as to release or allow the release of a therapeutic polypeptide therefrom at a desired lapsed time after the microparticle has been administered to a subject. For example, the polymer can be selected to release or allow the release of the therapeutic polypeptide prior to the therapeutic polypeptide beginning to diminish its activity, as the therapeutic polypeptide begins to diminish in activity, when the therapeutic polypeptide is partially diminished in activity, for example at least 25%, at least 50% or at least 75% diminished, when the therapeutic polypeptide is substantially diminished in activity, or when the therapeutic polypeptide is completely gone or no longer has activity.

Specific examples of suitable polymers that can be present in the microparticle polymer matrix include one or more of a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof.

Lactide-based polymers can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL- polymers. In addition to copolymers comprising glycolide and DL-lactide or L-lactide, copolymers of L-lactide and DL-lactide are commercially available. Homopolymers of lactide or glycolide are also commercially available.

In a particular aspect, when the biodegradable polymer is poly(lactide-co-glycolide), or a mixture of poly(lactide) and poly(glycolide), the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

In another aspect, the polymer can be a poly(caprolactone) or a poly(lactide-co-caprolactone). In one aspect, the polymer can be a poly(lactide-caprolactone), which, in various aspects, can be 95:5 poly(lactide-co-caprolactone), 85:15 poly(lactide-co-caprolactone), 75:25 poly(lactide-co- caprolactone), 65:35 poly(lactide-co-caprolactone), or 50:50 poly(lactide-co- caprolactone), where the ratios are mole ratios.

The microparticles of the invention can be prepared according to methods known in the art, for example the methods disclosed in U.S. Pat. No. 5,407,609 to Tice et al., which is hereby incorporated herein by reference in its entirety for its teachings of microparticle preparation methods.

The therapeutic polypeptide encapsulated within the biodegradable polymer matrix of the microparticle comprises at least 2 amino acids, which can be natural or nonnatural amino acids, wherein at least of the amino acids comprises one or more thiol (—SH) functional groups. In a further aspect, the therapeutic polypeptide comprises one or more cysteine or homocysteine residues.

The amount of the therapeutic polypeptide in the micorparticle can vary but will generally range from about 0.1% to about 80% by weight of the microparticle, preferably from about 0.1% to about 40%, and more preferably from about 5% to about 25% by weight of the microparticle.

In one aspect, the therapeutic polypeptide comprises at least two cysteine or homocysteine residues that form a disulfide bond. A specific example is a cyclic peptide that comprises two cysteine residues that form a loop. The redox reagent discussed below can moderate disulfide exchange for such a peptide and mitigate intermolecular disulfide bond formation while allowing the desired intramolecular disulfide bond to remain.

A wide variety of polypeptides can be used with the present invention. The polypeptide can comprise any number of amino acids, for example from about 2 to about 60 amino acids. Specific examples of suitable polypeptides include without limitation calcitonin, which has 32 amino acid residues including two cysteine residues, insulin, which has 51 amino acid residues, both forms of somatostatin, one of which has 14 amino acid residues, the other of which has 28 amino acid residues, octreotide, with is an 8 amino acid residue cyclic polypeptide, amylin, which has 37 amino acid residues, oxytocin, which has 9 amino acid residues, calcitonin gene related peptide (CGRP), which has 32 amino acid residues, and melanin concentration hormone (MCH), which is a 19 amino acid cyclic polypeptide.

The redox reagent of the microparticle is typically a small molecule compound, with a molecular weight generally less than about 1000 g/mol, preferably less than about 500 g/mol, and having at least one thiol (SH) functional group which can act as a reducing or oxidizing agent for disulfide bonds, thiols, or thiolate species present on the therapeutic polypeptide and thereby moderate disulfide exchange reactions among the polypeptide and enable the desired release of the polypeptide from the microparticle.

A wide variety of redox reagents can be used, but preferred agents are those that are approved for administration to a subject. Preferred examples include dithiothreitol, mercaptoethanol, cysteine, homocysteine, methionine, and glutathione. Glutathione, for example, is approved for intraocular administrations and therefore can be used in microparticle compositions that are to be administered intraocularly.

The redox reagent can function several ways in the microparticle. In one aspect, it is believed that the redox reagent can reduce the formation of undesirable intermolecular disulfide bonds between the therapeutic oligopeptide and thereby reduce the formation of aggregated polypeptides that are less likely to be released from the microparticle. In another aspect, it is believed that the redox reagent can allow intramolecular disulfide bonds (e.g., a disulfide bond holding a cyclic polypeptide together) to reform if they are broken through disulfide exchange reactions. Thus, in this aspect, the therapeutic polypeptide which has a native intramolecular disulfide bond is released in its desired form (i.e. with the intramolecular disulfide bond intact) due to the redox mediation accomplished by the redox reagent present in the microparticle.

The microparticles of the invention are useful as drug-delivery vehicles which can be administered to a subject to treat a variety of disorders. The microparticles can be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

In addition to the treatment of warm-blooded animals such as mice, rats, horses, cattle, sheep, dogs, cats, monkeys, etc., the microparticles of the invention are effective for use in humans. In general, pharmaceutical compositions comprising the microparticles can be prepared by uniformly and intimately bringing the microparticle into association with a liquid carrier or a finely divided solid carrier or both, and then, if necessary, shaping the product into the desired formulation. In the pharmaceutical composition the therapeutic polypeptide is included in an amount sufficient to produce the desired effect upon the process or condition of diseases. Accordingly, the pharmaceutical compositions of the present invention encompass any composition made by admixing a microparticle of the present invention and a pharmaceutically acceptable carrier, which can in some instances be the microparticle itself.

In practice, the microparticles of the invention can be combined as the active ingredient delivery vehicle in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. The pharmaceutical compositions can be prepared by any of the methods of pharmacy

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Optionally, tablets can be coated by standard aqueous or nonaqueous techniques.

A tablet containing the microparticle composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Pharmaceutical compositions comprising the microparticles of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions comprising the microparticles of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions comprising the microparticles of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In addition to the aforementioned carrier ingredients of the microparticle compositions, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a microparticle of the invention can also be prepared in powder or liquid concentrate form.

In the treatment of many disorders for which the microparticle compositions can be effective, an appropriate dosage level of the therapeutic polypeptide will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated.

The dosage regimens can be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In a specific aspect, the microparticles of the invention can be useful for the delivery of drugs in a targeted fashion through delivery directly into the eye, such as for example by injection. The microparticle can be so delivered to the anterior chamber of the eye or the posterior chamber of the eye including for example the vitreous, intraretinal space, subretinal space, intrachoroidal space and the suprachoroidal space.

Generally, the subject to which the microparticle can be administered is one that is in need of a desired therapeutic effect, such as the amelioration of a disorder present in the subject, for example, after being diagnosed with the disorder. The subject can be a vertebrate, such as a mammal, a fish, a bird, a reptile, or an amphibian. Thus, the subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

What is claimed is:

1. A polymer microparticle comprising a biodegradable polymer matrix, a therapeutic polypeptide comprising at least one natural or non-natural thiol (—SH) containing amino acid; and a redox reagent comprising at least one thiol (—SH) residue.

2. The microparticle of claim 1, wherein the therapeutic polypeptide and the redox reagent are encapsulated by the biodegradable polymer matrix.

3. The microparticle of claim 1, wherein the biodegradable polymer matrix comprises poly(lactide), poly(glycolide), poly(caprolactone), or a copolymer thereof.

4. The microparticle of claim 1, wherein the biodegradable polymer matrix comprises poly(lactide-co-glycolide).

5. The microparticle of claim 1, wherein the therapeutic polypeptide comprises one or more cysteine or homocysteine residues.

6. The microparticle of claim 1, wherein the therapeutic polypeptide is present in an amount ranging from about 0.1% to about 80% by weight of the microparticle.

7. The microparticle of claim 1, wherein the therapeutic polypeptide is present in an amount ranging from 1 to 40% by weight of the microparticle.

8. The microparticle of claim 1, wherein the therapeutic polypeptide is present in amount ranging from 5 to 25% by weight of the microparticle.

9. The microparticle of claim 1, wherein the redox reagent is glutathione, cysteine, homocysteine, dithiothreitol, mercaptoethanol, or methionine.

10. The microparticle of claim 1, wherein the redox reagent is present in an amount ranging from 0.1 to 10% by weight of the microparticle.

11. The microparticle of claim 1, wherein the redox reagent is present in an amount ranging from 0.1 to 5% by weight of the microparticle.

12. A method for delivering a therapeutic polypeptide comprising at least one natural or non-natural thiol (—SH) containing amino acid to a subject, the method comprising administering a polymer microparticle comprising a biodegradable polymer matrix, the therapeutic polypeptide, and a redox reagent comprising at least one thiol (—SH) residue, to the subject.

13. The method of claim 12, wherein the therapeutic polypeptide and the redox reagent are encapsulated by the biodegradable polymer matrix.

14. The method of claim 12, wherein the biodegradable polymer matrix comprises poly(lactide), poly(glycolide), poly(caprolactone), or a copolymer thereof.

15. The method of claim 12, wherein the biodegradable polymer matrix comprises poly(lactide-co-glycolide).

16. The method of claim 12, wherein the therapeutic polypeptide comprises one or more cysteine or homocysteine residues.

17. The method of claim 12, wherein the therapeutic polypeptide is present in an amount ranging from about 0.1% to about 80% by weight of the microparticle.

18. The method of claim 12, wherein the therapeutic polypeptide is present in an amount ranging from 1 to 40% by weight of the microparticle.

19. The method of claim 12, wherein the therapeutic polypeptide is present in amount ranging from 5 to 25% by weight of the microparticle.

20. The method of claim 12, wherein the redox reagent is glutathione, cysteine, homocysteine, dithiothreitol, mercaptoethanol, or methionine.

21. The method of claim 12, wherein the redox reagent is present in an amount ranging from 0.1 to 10% by weight of the microparticle.

22. The method of claim 12, wherein the redox reagent is present in an amount ranging from 0.1 to 5% by weight of the microparticle.
